(19) United States
(12) Patent Application Publication
(10) Pub. No.: US 2005/0049554 A1
(43) Pub. Date: Mar. 3, 2005

(54) Breakable syringe

(76) Inventor: Chih-Hsin Chueh, Taipei (TW)

Correspondence Address: ALAN D. KAMRATH;NIKOLAI & MERSEREAU, P.A., 820 INTERNATIONAL CENTRE, 900 SECOND AVENUE SOUTH, MINNEAPOLIS, MN, 55402-3325, US

(21) Appl. No.: 10/652,829

(22) Filed: Aug. 29, 2003

(51) Int. Cl. A61M 5/00

(52) U.S. Cl. 604/110

(57) ABSTRACT

A breakable syringe includes a barrel, a connecting member, a needle, and a plunger. Thus, the needle can be retracted into the barrel after use, thereby preventing the user or other person from being hurt by the protruding needle, so as to provide a safety effect. In addition, the plunger can be broken, so that the needle is stored in the barrel, thereby achieving the safe and sanitary effect.

### BACKGROUND OF THE INVENTION

1. Field of the Invention

The present invention relates to a breakable syringe, and more particularly to a breakable syringe that can be retracted inward by rotation.

2. Description of the Related Art

A conventional syringe comprises a barrel, a needle secured on an end of the barrel, and a plunger inserted into the barrel. When in use, the plunger is pushed forward to drain the air contained in the barrel outward to the ambient environment. Then, the plunger is pulled backward to draw the liquid medicine into the barrel. Thus, the plunger can be pushed forward to inject the liquid medicine through the needle into the human body. However, the needle is protruded outward from the barrel, so that the user or other person is easily hurt by the protruding needle after the syringe is used.

### SUMMARY OF THE INVENTION

The primary objective of the present invention is to provide a breakable syringe that can be retracted inward by rotation.

Another objective of the present invention is to provide a breakable syringe, wherein the needle and the needle cannula can be retracted into the barrel after use, thereby preventing the user or other person from being hurt by the protruding needle, so as to provide a safety effect.

A further objective of the present invention is to provide a breakable syringe, wherein the plunger can be broken, so that the needle cannula of the needle is stored in the barrel, thereby achieving the safe and sanitary effect.

A further objective of the present invention is to provide a breakable syringe that can be assembled easily and conveniently, thereby facilitating the user using the breakable syringe.

A further objective of the present invention is to provide a breakable syringe that has a rigid structure without detachment, so that the user can operate the breakable syringe safely.

In accordance with the present invention, there is provided a breakable syringe, comprising a barrel, a connecting member, a needle, and a plunger, wherein:

the barrel has an end formed with a through hole;

the connecting member is movably mounted in the barrel and has a first end formed with a lug protruded outward from the through hole of the barrel, and a second end formed with a nozzle;

the needle is mounted on the barrel and secured on the connecting member;

the plunger has a first end inserted into the barrel and formed with a piston movably mounted in the barrel; and

the piston of the plunger has a side formed with a reduced protrusion inserted into the nozzle of the connecting member.

Further benefits and advantages of the present invention will become apparent after a careful reading of the detailed description with appropriate reference to the accompanying drawings.

### BRIEF DESCRIPTION OF THE DRAWINGS

FIG. 1 is an exploded perspective view of a breakable syringe in accordance with the preferred embodiment of the present invention;

FIG. 1A is a partially cross-sectional view of the connecting member of the breakable syringe as shown in FIG. 1;

FIG. 2 is a partially plan cross-sectional assembly view of the breakable syringe as shown in FIG. 1;

FIG. 3 is a schematic operational view of the breakable syringe as shown in FIG. 2;

FIG. 4 is a schematic operational view of the breakable syringe as shown in FIG. 3;

FIG. 5 is a schematic operational view of the breakable syringe as shown in FIG. 4;

FIG. 6 is a schematic operational view of the breakable syringe as shown in FIG. 5;

FIG. 7 is a schematic operational view of the breakable syringe as shown in FIG. 6; and

FIG. 8 is a schematic operational view of the breakable syringe as shown in FIG. 6.

### DETAILED DESCRIPTION OF THE INVENTION

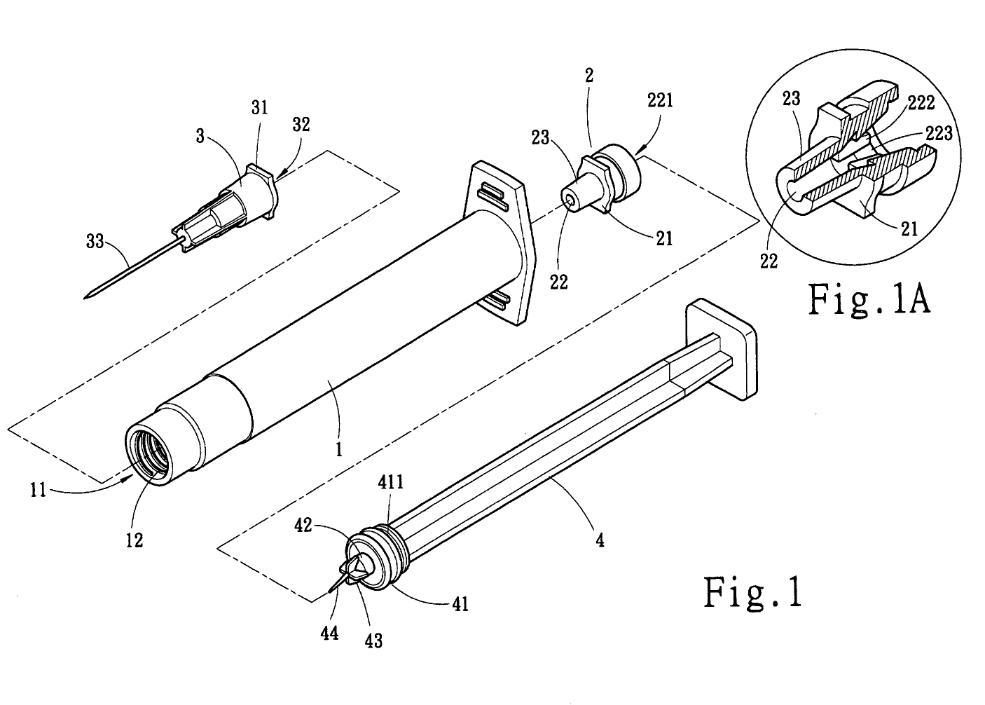

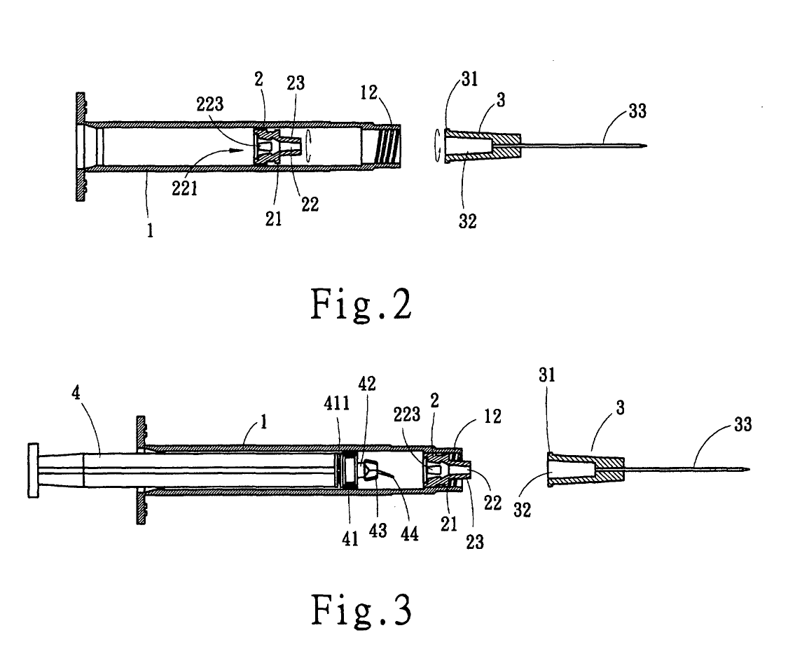

Referring to the drawings and initially to FIGS. 1 and 2, a breakable syringe in accordance with the preferred embodiment of the present invention comprises a barrel 1, a connecting member 2, a needle 3, and a plunger 4.

The barrel 1 has an end formed with a through hole 11 having an inner periphery formed with an inner thread 12.

As shown in FIGS. 1 and 1A, the connecting member 2 is movably mounted in the barrel 1 and has a first end formed with a gradually reduced tapered lug 23 protruded outward from the through hole 11 of the barrel 1, a mediate portion formed with an enlarged flange 21, and a second end formed with a gradually expanded nozzle 221. The enlarged flange 21 of the connecting member 2 is engaged into the inner thread 12 of the barrel 1. The lug 23 of the connecting member 2 is formed with a through hole 22. The nozzle 221 of the connecting member 2 has an inner wall formed with a plurality of tapered protruding teeth 222 and a plurality of tapered guide grooves 223 located between the protruding teeth 222.

The needle 3 is mounted on the barrel 1 and secured on the connecting member 2. The needle 3 has a first end formed with a tapered recess 32 to receive the lug 23 of the connecting member 2. The first end of the needle 3 has a periphery formed with an enlarged flange 31 engaged into the inner thread 12 of the barrel 1. The needle 3 has a second end provided with a needle cannula 33 communicating with the recess 32.

The plunger 4 has a first end inserted into the barrel 1 and formed with a piston 41 movably mounted in the barrel 1. The plunger 4 has a second end protruded outward from the barrel 1. The piston 41 of the plunger 4 has a first side formed with an annular groove 411 detachably connected to the first end of the plunger 4 and a second side formed with a reduced protrusion 42 inserted into the nozzle 221 of the connecting member 20. The protrusion 42 of the plunger 4 has a periphery formed with a plurality of tapered protruding teeth 43 inserted into the guide grooves 223 of the connecting member 2 and engaged with the protruding teeth 222 of the connecting member 2. The tapered protruding teeth 43 of the plunger 4 are arranged in a radiating manner. The protrusion 42 of the plunger 4 has an end formed with an elastic press bar 44 extended into the through hole 22 of the lug 23 of the connecting member 2. The elastic press bar 44 is arranged in an oblique manner.

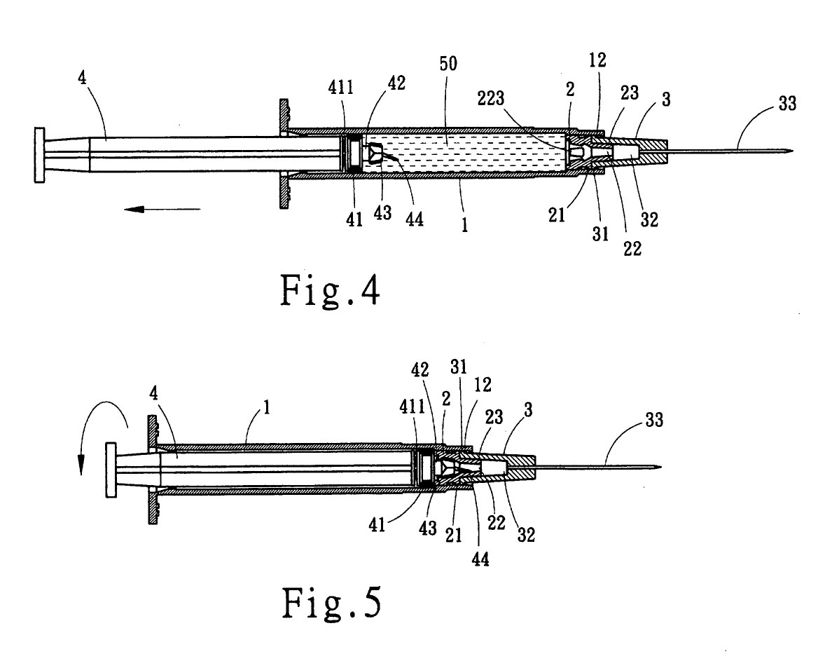

In assembly, the connecting member 2 is movable in the barrel 1 and the enlarged flange 21 of the connecting member 2 is engaged into the inner thread 12 of the barrel 1, so that the lug 23 of the connecting member 2 is protruded outward from the through hole 11 of the barrel 1 as shown in FIG. 3. Then, the piston 41 of the plunger 4 is inserted into the barrel 1. Then, the enlarged flange 31 of the needle 3 is engaged into the inner thread 12 of the barrel 1, so that the lug 23 of the connecting member 2 is inserted into the tapered recess 32 of the needle 3 as shown in FIG. 4.

When in use, the piston 41 of the plunger 4 is pushed forward to drain the air contained in the barrel 1 outward to the ambient environment. Then, the piston 41 of the plunger 4 is pulled backward to draw the liquid medicine 50 into the barrel 1 as shown in FIG. 4. Then, after the liquid medicine 50 is completely injected outward from the barrel 1, the plunger 4 is entirely inserted into the barrel 1, so that the tapered protruding teeth 43 of the plunger 4 are inserted into the guide grooves 223 of the connecting member 2 and engaged with the protruding teeth 222 of the connecting member 2, while the elastic press bar 44 of the plunger 4 is extended into the through hole 22 of the lug 23 of the connecting member 2 as shown in FIG. 5.

At this time, the lug 23 of the connecting member 2 is closely forced into the tapered recess 32 of the needle 3, thereby forming a vacuum state, so that the connecting member 2 is closely combined with the needle 3 without detachment.

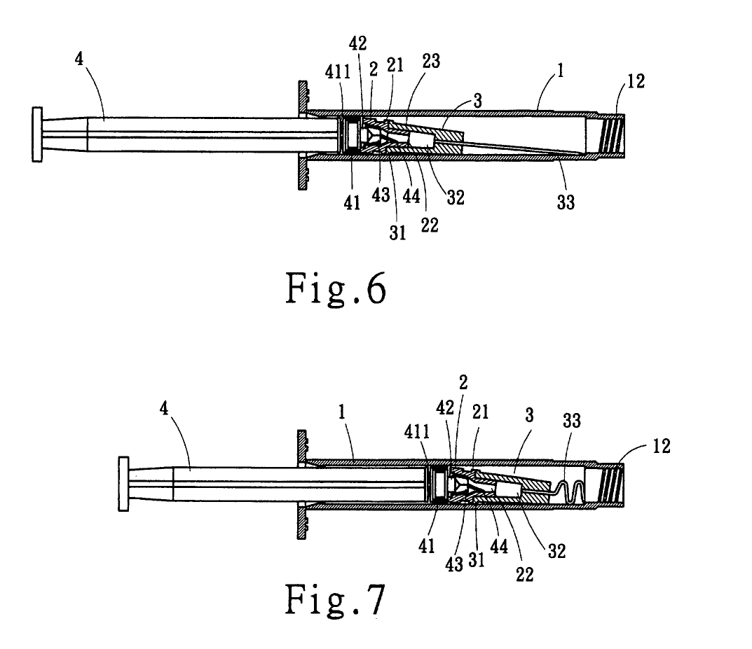

Then, the plunger 4 is rotated as shown in FIG. 5, so that the connecting member 2 and the needle 3 are rotated by rotation of the plunger 4 to detach from the inner thread 12 of the barrel 1. Then, the plunger 4 is pulled backward to retract the needle 3 and the needle cannula 33 into the barrel 1 as shown in FIG. 6.

At this time, the elastic press bar 44 of the plunger 4 is rested on the inner wall of the through hole 22 of the lug 23 of the connecting member 2, so that the connecting member 2 is pressed in an oblique manner and the needle cannula 33 of the needle 3 is also pressed in an oblique manner to abut the wall of the inner thread 12 of the barrel 1 as shown in FIG. 6.

Then, the plunger 4 is pushed forward into the barrel 1 as shown in FIG. 7, thereby bending and deforming the needle cannula 33 of the needle 3.

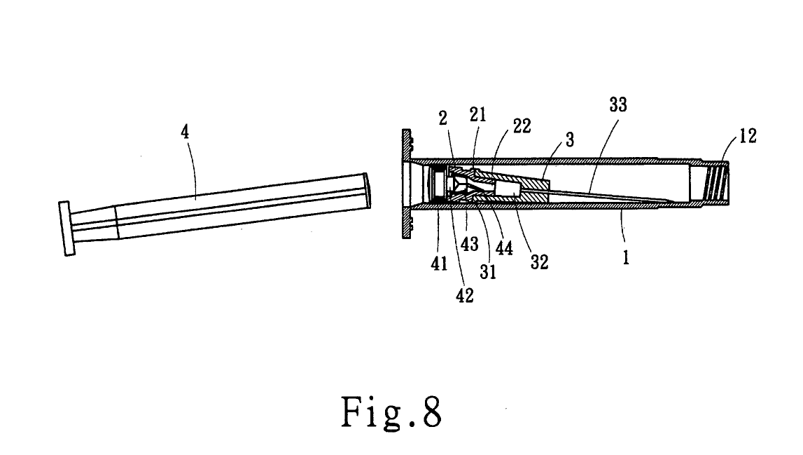

Alternatively, the piston 41 of the plunger 4 is detached from the plunger 4 at the position of the annular groove 411 and is urged in the inner wall of the barrel 1 as shown in FIG. 8, so that the needle cannula 33 of the needle 3 is stored in the barrel 1.

Accordingly, the plunger 4 can be broken, so that the needle cannula 33 of the needle 3 is stored in the barrel 1, thereby achieving the safe and sanitary effect. In addition, the breakable syringe can be assembled easily and conveniently, thereby facilitating the user using the breakable syringe. Further, the breakable syringe has a rigid structure without detachment, so that the user can operate the breakable syringe safely.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the true scope of the invention.

1. A breakable syringe, comprising a barrel, a connecting member, a needle, and a plunger, wherein:
the barrel has an end formed with a through hole;
the connecting member is movably mounted in the barrel and has a first end formed with a lug protruded outward from the through hole of the barrel, and a second end formed with a nozzle;
the needle is mounted on the barrel and secured on the connecting member;
the plunger has a first end inserted into the barrel and formed with a piston movably mounted in the barrel; and
the piston of the plunger has a side formed with a reduced protrusion inserted into the nozzle of the connecting member.

2. The breakable syringe in accordance with claim 1, wherein the lug of the connecting member has a gradually reduced tapered shape.

3. The breakable syringe in accordance with claim 1, wherein the nozzle of the connecting member has a gradually expanded shape.

4. The breakable syringe in accordance with claim 1, wherein the through hole of the barrel has an inner periphery formed with an inner thread, and the connecting member has a mediate portion formed with an enlarged flange engaged into the inner thread of the barrel.

5. The breakable syringe in accordance with claim 1, wherein the lug of the connecting member is formed with a through hole.

6. The breakable syringe in accordance with claim 1, wherein the nozzle of the connecting member has an inner wall formed with a plurality of tapered protruding teeth and a plurality of tapered guide grooves located between the protruding teeth, and the protrusion of the plunger has a periphery formed with a plurality of tapered protruding teeth inserted into the guide grooves of the connecting member and engaged with the protruding teeth of the connecting member.

7. The breakable syringe in accordance with claim 6, wherein the tapered protruding teeth of the plunger are arranged in a radiating manner.

8. The breakable syringe in accordance with claim 1, wherein the needle has a first end formed with a tapered recess to receive the lug of the connecting member.

9. The breakable syringe in accordance with claim 8, wherein the through hole of the barrel has an inner periphery formed with an inner thread, and the first end of the needle has a periphery formed with an enlarged flange engaged into the inner thread of the barrel.

10. The breakable syringe in accordance with claim 8, wherein the needle has a second end provided with a needle cannula communicating with the recess.

11. The breakable syringe in accordance with claim 1, wherein the piston of the plunger is formed with an annular groove detachably connected to the first end of the plunger.

12. The breakable syringe in accordance with claim 1, wherein the protrusion of the plunger has an end formed with an elastic press bar extended into the through hole of the lug of the connecting member.

13. The breakable syringe in accordance with claim 12, wherein the elastic press bar is arranged in an oblique manner.

14. The breakable syringe in accordance with claim 1, wherein the plunger has a second end protruded outward from the barrel.

15. The breakable syringe in accordance with claim 8, wherein the lug of the connecting member is closely forced into the tapered recess of the needle, thereby forming a vacuum state, so that the connecting member is closely combined with the needle without detachment.
